(19) United States
(12) Patent Application Publication
(10) Pub. No.: US 2022/0273662 A1
(43) Pub. Date: Sep. 1, 2022

(54) USE OF SILDENAFIL AND ROCK INHIBITORS FOR TREATING STROKE OR SEQUELAE FOLLOWING STROKE

(71) Applicant: SOONCHUNHYANG UNIVERSITY INDUSTRY ACADEMY COOPERATION FOUNDATION, Asan-si (KR)

(72) Inventors: Jae-Sang OH, Asan-si (KR); Duk-Soo KIM, Cheonan-si (KR); Man-Ryul LEE, Cheonan-si (KR); Juhyeon KANG, Seoul (KR)

(21) Appl. No.: 17/744,162

(22) Filed: May 13, 2022

Related U.S. Application Data
(63) Continuation of application No. PCT/KR2019/015662, filed on Nov. 15, 2019.

(30) Foreign Application Priority Data
Nov. 14, 2019 (KR) 10-2019-0146217
Nov. 14, 2019 (KR) 10-2019-0146222
Nov. 14, 2019 (KR) 10-2019-0146223
Nov. 14, 2019 (KR) 10-2019-0146225

(51) Int. Cl. A61K 31/519; A61K 31/4439; A61P 9/10

(52) U.S. Cl. CPC A61K 31/519; A61K 31/4439; A61P 9/10

(57) ABSTRACT

The present invention relates to a use of a pharmaceutical composition for treating stroke or sequelae following stroke, the composition comprising a phosphodiesterase type 5 activity inhibitor and a Rho-associated kinase (ROCK) inhibitor.

The present inventors have performed research, based on clinical knowledge related to stroke treatment, in order to use sildenafil and a ROCK inhibitor in combination to treat stroke or sequelae caused by stroke, and as a result, confirmed for the first time that stroke or sequelae caused by stroke were significantly improved when sildenafil and a ROCK inhibitor were co-administered compared to when sildenafil or the ROCK inhibitor was administered by itself. Therefore, it is expected that a pharmaceutical composition according to the present invention will be able to be effectively used for treating or studying stroke or stroke-related sequelae.

### TECHNICAL FIELD

The present invention relates to a use of a pharmaceutical composition for treating stroke or sequelae following stroke, the composition comprising a phosphodiesterase type 5 activity inhibitor and a Rho-associated kinase (ROCK) inhibitor.

### BACKGROUND ART

Stroke is a neurological symptom that appears when a blood vessel that supplies blood to the brain is blocked or ruptures and the brain tissue in that area is damaged, and is roughly divided into two types: ischemic stroke and hemorrhagic stroke. Among them, ischemic stroke occurs when the brain tissue becomes ischemic due to a decrease in or blockage of supply of blood to the brain tissue, hemorrhagic stroke occurs due to hemorrhage when a blood vessel ruptures, and it is reported that ischemic stroke accounts for about 80% of all stroke patients.

When ischemic stroke occurs, cells cannot obtain oxygen and nutrients due to temporary interruption of blood flow, but reperfusion, which is a measure to resume vascular flow using surgery or a thrombolytic agent, enables reoxygenation. Although reperfusion is essential for the recovery process in that it provides cells with oxygen and nutrients, resumption of vascular flow with a damaged blood-brain barrier produces excess oxygen compounds, that is, reactive oxygen species (ROS), and may also lead to severe cerebral edema or conversion to cerebral hemorrhage, while being accompanied by an inflammatory response. Time is the most important in the treatment of stroke, because brain cells die once a blood vessel is blocked, killing millions of cells per minute. However, recent studies show that different patients have different prognoses even when the same site is blocked. As a result of analyzing patients with a good prognosis, it was reported that when the cerebral blood vessels in the brains of the patients are blocked, a self-protective function dilates the surrounding cerebral blood vessels and at the same time helps blood flow in the blocked area. Therefore, at the onset of cerebral infarction, it can be seen that the dilatation function of peripheral blood vessels makes a prognosis for patients different.

Stroke is a temporary brain disease that occurs because blood vessels to the brain are blocked or rupture, and has serious sequelae, and brain nerve cell damage causes various neurological symptoms depending on the site and extent of bleeding and closure, such as loss of locomotor activity, paresthesia, cognitive impairment, speech impairment, loss of consciousness, and impaired swallowing function.

Further, causes of brain nerve cell damage that occur due to stroke include the release of excessive excitatory neurotransmitters, production of free radicals, inhibition of protein synthesis, dysfunction of gene expression, activation of immune responses, and the like, and due to the complexity of the mechanism of brain nerve cell damage, no therapeutic agent capable of protecting against damage to brain nerve cells has yet been developed.

Meanwhile, sildenafil is a drug used to treat erectile dysfunction and pulmonary hypertension, and has an effect of ameliorating erectile dysfunction and improving pulmonary artery pressure by relaxing the smooth muscles distributed in the corresponding organ to facilitate the supply of blood, and it is known that 20 mg tablets are used to treat pulmonary hypertension and 25 mg, 50 mg and 100 mg tablets are used to treat erectile dysfunction.

In addition, a Rho-associated kinase (ROCK) inhibitor is a material having a function of suppressing apoptosis, and is known to have functions, such as neurocladism, myosin phosphorylation, and suppression of agonist-induced Ca²⁺ sensitization in smooth muscle contraction. More specifically, the ROCK inhibitor has been reported to have functions of alleviating the abnormal structure of muscle cells that cause hypertension and asthma, increasing blood flow in the optic disc, and continuously reducing intraocular pressure.

Although there are studies on the therapeutic use of ROCK inhibitors for inflammatory diseases (Korean Patent No. 10-1794009) and studies on the use of sildenafil for skeletal muscle regeneration and enhancement (Korean Patent No. 10-1852358), there are no studies on the use of a pharmaceutical composition including a phosphodiesterase type 5 activity inhibitor and a ROCK inhibitor to treat stroke or the sequelae following stroke.

The details described as the aforementioned background art are only for the purpose of improving the understanding of the background of the present invention, and should not be taken as acknowledging that they correspond to the related art already known to those skilled in the art.

### DISCLOSURE

### Technical Problem

As a result of performing research to use a phosphodiesterase type 5 activity inhibitor in combination with a ROCK inhibitor for the treatment of stroke or various sequelae following stroke based on clinical knowledge related to stroke treatment, the present inventors confirmed that when sildenafil and the ROCK inhibitor were co-administered, the size of brain damage, loss of locomotor activity, an anxiety disorder, and cognitive function decline due to stroke were significantly ameliorated compared to when sildenafil and the ROCK inhibitor were each administered alone, thereby completing the present invention based on this.

Therefore, an object of the present invention is to provide a pharmaceutical composition for treating an ischemic cerebrovascular disease or sequelae following the ischemic cerebrovascular disease, including: a phosphodiesterase type 5 activity inhibitor; and a Rho-associated kinase (ROCK) inhibitor.

Further, another object of the present invention is to provide a pharmaceutical composition for improving the prognosis of an ischemic cerebrovascular disease, including: a phosphodiesterase type 5 activity inhibitor; and a Rho-associated kinase (ROCK) inhibitor.

In addition, still another object of the present invention is to provide a pharmaceutical composition for enhancing a cognitive function, including: a phosphodiesterase type 5 activity inhibitor; and a Rho-associated kinase (ROCK) inhibitor.

However, technical problems to be solved by the present invention are not limited to the aforementioned problems, and other problems that are not mentioned may be clearly understood by those skilled in the art from the following description.

### Technical Solution

To achieve the objects of the present invention as described above, the present invention provides a pharmaceutical composition for treating an ischemic cerebrovascular disease or sequelae following the ischemic cerebrovascular disease, including: a phosphodiesterase type 5 activity inhibitor; and a Rho-associated kinase inhibitor.

As an exemplary embodiment of the present invention, the ischemic cerebrovascular disease may be any one selected from the group consisting of ischemic stroke (cerebral infarction), thrombosis, embolism, transient ischemic attack, leukoplakia and small infarction.

As another exemplary embodiment of the present invention, the sequelae following the ischemic cerebrovascular disease may be any one selected from the group consisting of loss of locomotor activity, an anxiety disorder and cognitive function decline.

As still another exemplary embodiment of the present invention, the phosphodiesterase type 5 activity inhibitor may be any one selected from the group consisting of mirodenafil, sildenafil, vardenafil, tadalafil, udenafil, dasantafil, avanafil and pharmaceutically acceptable salts, solvates and hydrates thereof.

As yet another exemplary embodiment of the present invention, the ROCK inhibitor may be any one selected from the group consisting of fasudil, ripasudil, RKI-1447, Y-27632, GSK429286A, Y-30141 and pharmaceutically acceptable salts, solvates and hydrates thereof.

As yet another exemplary embodiment of the present invention, the pharmaceutical composition may be for administration before reperfusion after cerebrovascular occlusion, simultaneously with reperfusion or after reperfusion.

Furthermore, the present invention provides a method for treating an ischemic cerebrovascular disease or sequelae following the ischemic cerebrovascular disease, the method including administering the pharmaceutical composition to an individual.

Further, the present invention provides a use of the pharmaceutical composition for treating an ischemic cerebrovascular disease or sequelae following the ischemic cerebrovascular disease.

In addition, the present invention provides a pharmaceutical composition for improving the prognosis of an ischemic cerebrovascular disease, including: a phosphodiesterase type 5 activity inhibitor; and a Rho-associated kinase (ROCK) inhibitor.

Furthermore, the present invention provides a method for improving the prognosis of an ischemic cerebrovascular disease, the method including administering the pharmaceutical composition to an individual.

Further, the present invention provides a use of the pharmaceutical composition for improving the prognosis of an ischemic cerebrovascular disease.

In addition, the present invention provides a method for enhancing a cognitive function, including: a phosphodiesterase type 5 activity inhibitor; and a Rho-associated kinase (ROCK) inhibitor.

Furthermore, the present invention provides a method for enhancing a cognitive function, the method including administering the pharmaceutical composition to an individual.

Further, the present invention provides a use of the pharmaceutical composition for enhancing a cognitive function.

### Advantageous Effects

The present inventors have performed research, based on clinical knowledge related to stroke treatment, in order to use sildenafil and a ROCK inhibitor in combination to treat stroke or sequelae caused by stroke, and as a result, confirmed for the first time that stroke or sequelae caused by stroke were significantly improved when sildenafil and a ROCK inhibitor were co-administered compared to when sildenafil or the ROCK inhibitor was administered by itself. Therefore, it is expected that a pharmaceutical composition according to the present invention will be able to be effectively used for treating or studying stroke or stroke-related sequelae.

### DESCRIPTION OF DRAWINGS

FIG. 1 illustrates the results of confirming the size of stroke-induced brain damage in a control, a stroke (cerebral infarction) animal model, a drug-alone treatment group, and a drug combination treatment group in a brain slice image. Here, MCAo30 represents a stroke (cerebral infarction) animal model, SIL represents a sildenafil-alone treatment group, ROCK-I represents a Rho-associated kinase (ROCK) inhibitor-alone treatment group, SR represents a combination treatment group of sildenafil and a Rho-associated kinase (ROCK) inhibitor, pre represents an administration group before reperfusion after middle cerebral artery occlusion, and post represents an administration group after reperfusion after middle cerebral artery occlusion.

FIG. 2 is a graph illustrating the results of confirming the reduction level of direct damage to the brain (infarction volume) due to stroke in a control, a stroke (cerebral infarction) animal model, a drug-alone treatment group and a drug combination treatment group. Here, MCAo30 represents a stroke (cerebral infarction) animal model, SIL represents a sildenafil-alone treatment group, ROCK-I represents a Rho-associated kinase (ROCK) inhibitor-alone treatment group, SR represents a combination treatment group of sildenafil and a Rho-associated kinase (ROCK) inhibitor, pre represents an administration group before reperfusion after middle cerebral artery occlusion, and post represents an administration group after reperfusion after middle cerebral artery occlusion.

FIG. 3 illustrates the tracing figures for confirming the locomotor activity levels of a control, a stroke (cerebral infarction) animal model, a drug-alone treatment group and a drug combination treatment group. Here, MCAo30 represents a stroke (cerebral infarction) animal model, SIL represents a sildenafil-alone treatment group, ROCK-I represents a Rho-associated kinase (ROCK) inhibitor-alone treatment group, SR represents a combination treatment group of sildenafil and a Rho-associated kinase (ROCK) inhibitor, pre represents an administration group before reperfusion after middle cerebral artery occlusion, and post represents an administration group after reperfusion after middle cerebral artery occlusion.

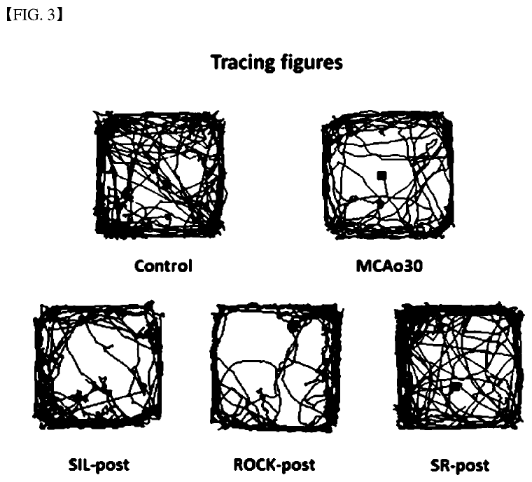

FIG. 4 is a graph illustrating the results of confirming the amelioration levels of loss of locomotor activity in a control, a stroke (cerebral infarction) animal model, a drug-alone treatment group and a drug combination treatment group. Here, MCAo30 represents a stroke (cerebral infarction) animal model, SIL represents a sildenafil-alone treatment group, ROCK-I represents a Rho-associated kinase (ROCK) inhibitor-alone treatment group, and SR represents a combination treatment group of sildenafil and a Rho-associated kinase (ROCK) inhibitor.

FIG. 5 illustrates the light-dark box test results for confirming the anxiety levels of a control, a stroke (cerebral infarction) animal model, a drug-alone treatment group and a drug combination treatment group. Here, MCAo30 represents a stroke (cerebral infarction) animal model, SIL represents a sildenafil-alone treatment group, ROCK-I represents a Rho-associated kinase (ROCK) inhibitor-alone treatment group, and SR represents a combination treatment group of sildenafil and a Rho-associated kinase (ROCK) inhibitor.

FIG. 6 illustrates an escape hole used in a Barnes maze test.

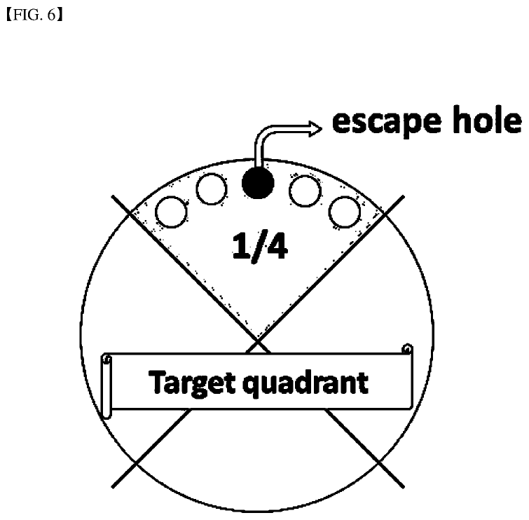

FIG. 7 illustrates the tracing figures for confirming the cognitive functions of a control, a stroke (cerebral infarction) animal model, a drug-alone treatment group and a drug combination treatment group. Here, MCAo30 represents a stroke (cerebral infarction) animal model, SIL represents a sildenafil-alone treatment group, ROCK-I represents a Rho-associated kinase (ROCK) inhibitor-alone treatment group, SR represents a combination treatment group of sildenafil and a Rho-associated kinase (ROCK) inhibitor, pre represents an administration group before reperfusion after middle cerebral artery occlusion, and post represents an administration group after reperfusion after middle cerebral artery occlusion.

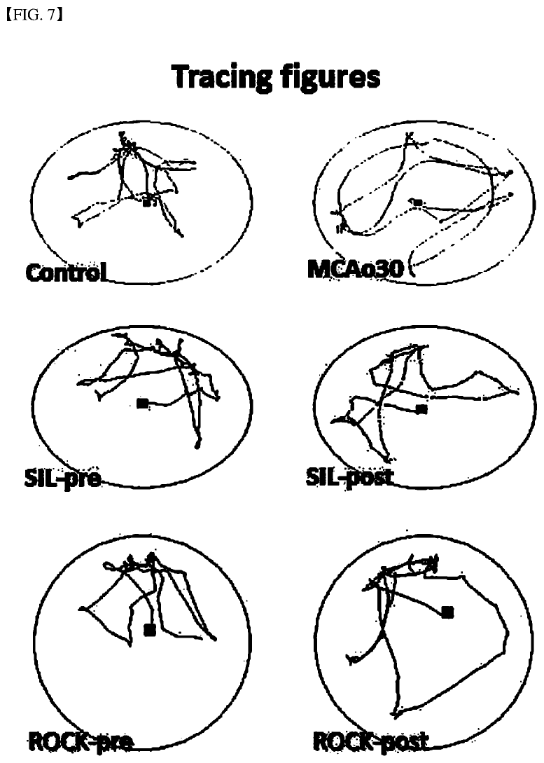

FIG. 8 illustrates the results of confirming the amelioration levels of cognitive function decline in a control, a stroke (cerebral infarction) animal model, a drug-alone treatment group and a drug combination treatment group in a graph. Here, MCAo30 represents a stroke (cerebral infarction) animal model, SIL represents a sildenafil-alone treatment group, ROCK-I represents a Rho-associated kinase (ROCK) inhibitor-alone treatment group, SR represents a combination treatment group of sildenafil and a Rho-associated kinase (ROCK) inhibitor, pre represents an administration group before reperfusion after middle cerebral artery occlusion, and post represents an administration group after reperfusion after middle cerebral artery occlusion.

FIG. 9 illustrates the results of confirming whether a decrease in synaptic plasticity due to stroke can be ameliorated by measuring a fEPSP slope in a control, a stroke (cerebral infarction) animal model, and a sildenafil-alone treatment group. Here, MCAo30 represents a stroke (cerebral infarction) animal model, SIL represents a sildenafil-alone treatment group, pre represents an administration group before reperfusion after middle cerebral artery occlusion, and post represents an administration group after reperfusion after middle cerebral artery occlusion.

FIG. 10 illustrates the amelioration of a decrease in synaptic plasticity due to stroke by measuring a fEPSP slope in a control, a stroke (cerebral infarction) animal model, and a sildenafil-alone treatment group in a graph. Here, MCAo30 represents a stroke (cerebral infarction) animal model, SIL represents a sildenafil-alone treatment group, pre represents an administration group before reperfusion after middle cerebral artery occlusion, and post represents an administration group after reperfusion after middle cerebral artery occlusion.

### MODES OF THE INVENTION

As a result of performing research to use sildenafil and a ROCK inhibitor in combination for the treatment of stroke or sequelae due to stroke based on clinical knowledge related to stroke treatment, the present inventors confirmed that when sildenafil and the ROCK inhibitor were co-administered, the degree of brain damage, loss of locomotor activity, an anxiety disorder, and cognitive function decline were significantly ameliorated compared to when sildenafil and the ROCK inhibitor were each administered alone, thereby completing the present invention based on this.

Thus, provided is a pharmaceutical composition for treating an ischemic cerebrovascular disease or sequelae following the ischemic cerebrovascular disease, the composition including: a phosphodiesterase type 5 activity inhibitor; and a Rho-associated kinase (ROCK) inhibitor.

An “ischemic cerebrovascular disease”, which is a disease to be targeted in the present invention, refers to a general term for diseases that occur because blood required for the brain is not supplied due to blockage of blood vessels in the brain for some reasons, is also referred to as a cerebral ischemia disease, and the ischemic cerebrovascular disease refers to a disease that occurs because oxygen and glucose are not supplied normally due to the reduction in blood flow to the brain, and as a result, the death of nerve cells in brain cells that are known to be sensitive to cerebral ischemia is induced, and examples of the ischemic cerebrovascular disease include ischemic stroke (cerebral infarction), thrombosis, embolism, transient ischemic attack, leukoplakia, small infarction, and the like, and may preferably be ischemic stroke, but is not limited thereto.

Examples of symptoms of “loss of locomotor activity following the ischemic cerebrovascular disease” which is a disease to be targeted in the present invention include facial palsy, loss of muscular strength of the arms (or the legs), dysarthria, and the like, but are not limited thereto.

Although anxiety symptoms proportional to a risk according to the actual threat or situation have been present for 6 months and any of the following three symptoms appears for “anxiety disorder following the ischemic cerebrovascular disease” which is a disease to be targeted in the present invention, the anxiety disorder is not limited thereto: feeling wound-up, tense, or restless; fatigue; difficulty in concentration; irritability; significant muscle tension; somnipathy. Further, as a result of a systematic review (39 cohort including 4,706 patients), it was confirmed that 24% of stroke patients had anxiety symptoms when assessed by a grading scale, and that 18% of stroke patients had an anxiety disorder during the first 5 years after stroke. Although the use of antidepressants to treat persistent, frequent and severe emotional instability is appropriate, an appropriate drug type, duration or dose is unknown.

Symptoms of “cognitive function decline following the ischemic cerebrovascular disease” which is a disease to be targeted in the present invention are very complicated and comprehensive symptoms related to perception, analysis, language, memory, and judgment, and exhibit various types of cognitive function decline depending on the position and degree of brain damage, and examples thereof include a decrease in learning capacity, spatial neglect, a decrease in visual spatial perception, attention degradation, memory degradation, and the like, but are not limited thereto.

In the present invention, a “phosphodiesterase type 5 activity inhibitor (PDE inhibitor)” is an enzyme found in various tissues. It is known that the PDE inhibitors provide strong inhibition of selected isoenzymes without the side effects that result from non-selective inhibitors, and PDE-5 inhibitors are used for the treatment of primary pulmonary hypertension and erectile dysfunction, and examples thereof include mirodenafil, sildenafil, vardenafil, tadalafil, udenafil, dasantafil, avanafil, and the like, and may preferably be sildenafil, but are not limited thereto.

In the present invention, a “Rho-associated kinase (ROCK) inhibitor” is a material having a function of suppressing apoptosis, and is known to have functions, such as neurocladism, myosin phosphorylation, and suppression of agonist-induced Ca2+ sensitization in smooth muscle contraction. More specifically, the ROCK inhibitor has been reported to have functions of alleviating the abnormal structure of muscle cells that cause hypertension and asthma, increasing blood flow in the optic disc, and continuously reducing intraocular pressure, and examples thereof include fasudil, ripasudil, RKI-1447, Y-27632, GSK429286A, Y-30141 and the like, and may preferably be N-(6-fluoro-1H-indazol-5-yl)-6-methyl-2-oxo-4-[4-(trifluoromethyl)phenyl]-3,4-dihydro-1H-pyridine-5-carboxamide (GS K429286A) represented by the following Chemical Formula 1, but are not limited thereto.

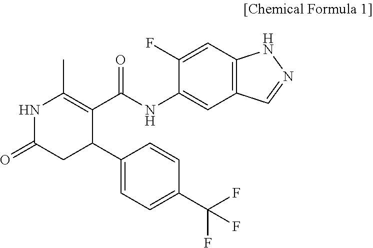 [SMILES: CC1=C(C(=O)Nc2cc3cn[nH]c3cc2F)C(c2ccc(C(F)(F)F)cc2)CC(=O)N1]

Further, the pharmaceutical composition may be for administration before reperfusion after cerebrovascular occlusion, simultaneously with reperfusion or after reperfusion, and may preferably be for administration reperfusion after middle cerebral artery occlusion, simultaneously with reperfusion or after reperfusion, but the administration is not limited thereto, and as used herein, the “reperfusion” refers to a measure to resume vascular flow using surgery or a thrombolytic agent when the supply of oxygen and nutrients is interrupted due to temporary interruption of blood flow in organs or tissues.

As described above, in an exemplary embodiment of the present invention, it was confirmed that when sildenafil (phosphodiesterase type 5 activity inhibitor) and GSK429286A (ROCK inhibitor) were co-administered in order to treat brain damage occurring in an acute stroke (cerebral infarction) animal model, direct damage to the brain (infarction volume) due to stroke was significantly reduced compared to when the sildenafil or GSK429286A was each administered alone to the animal model (see Example 2).

In addition, in another exemplary embodiment of the present invention, it was confirmed that when sildenafil (phosphodiesterase type 5 activity inhibitor) and GSK429286A (ROCK inhibitor) were co-administered in order to treat the loss of locomotor activity occurring in an acute stroke (cerebral infarction) animal model, the degree of the loss of locomotor activity was significantly ameliorated compared to when the sildenafil or GSK429286A was each administered alone to the animal model (see Example 3).

Furthermore, in still another exemplary embodiment of the present invention, it was confirmed that when sildenafil (phosphodiesterase type 5 activity inhibitor) and GSK429286A (ROCK inhibitor) were co-administered in order to treat an anxiety disorder occurring in an acute stroke (cerebral infarction) animal model, the degree of anxiety increased due to stroke was significantly ameliorated compared to when GSK429286A was administered alone to the animal model (see Example 4).

Further, in yet another exemplary embodiment of the present invention, it was confirmed that when sildenafil (phosphodiesterase type 5 activity inhibitor) and GSK429286A (ROCK inhibitor) were co-administered in order to treat cognitive function decline occurring in an acute stroke (cerebral infarction) animal model, the animals were observed to reach an escape hole at a level similar to the escape time of a normal control, and thus the cognitive function decline was significantly ameliorated, and it was confirmed that some individuals in the co-administration group reduced a period to reach an escape hole compared to a normal control, and thus showed an improved cognitive function (enhancement in memory) compared to a normal control in which stroke did not occur (see Example 5).

The results of the exemplary embodiments show the effects of effectively reducing brain damage occurring due to stroke when sildenafil (phosphodiesterase type 5 activity inhibitor) and GSK429286A (ROCK inhibitor) are co-administered compared to when sildenafil (phosphodiesterase type 5 activity inhibitor) or GSK429286A is each administered alone, mean that the loss of locomotor activity, an anxiety disorder, and cognitive function decline, which are sequelae due to stroke, are more effectively ameliorated, and thus, suggest that the pharmaceutical composition including sildenafil and GSK429286A according to the present invention can be usefully used as a therapeutic agent for stroke or sequelae thereof (loss of locomotor activity, an anxiety disorder, and cognitive function decline).

In addition, the results of the exemplary embodiments suggest that the pharmaceutical composition including sildenafil and GSK429286A can be usefully used as an enhancer of cognitive function because the reduced escape time when sildenafil (phosphodiesterase type 5 activity inhibitor) and GSK429286A (ROCK inhibitor) are co-administered compared to when sildenafil or GSK429286A is each administered alone means that the cognitive function is enhanced compared to a normal control.

The pharmaceutical composition according to the present invention includes: a phosphodiesterase type 5 activity inhibitor; and a Rho-associated kinase (ROCK) inhibitor) as active ingredients, and may also include a pharmaceutically acceptable carrier. The pharmaceutically acceptable carrier is typically used in formulation, and includes saline, sterile water, Ringer's solution, buffered saline, cyclodextrin, a dextrose solution, a maltodextrin solution, glycerol, ethanol, a liposome, and the like, but is not limited thereto, and may further include other typical additives such as an antioxidant and a buffer, if necessary. Further, the composition may be formulated into an injectable formulation, such as an aqueous solution, a suspension, and an emulsion, a pill, a capsule, granules, or a tablet by additionally adding a diluent, a dispersant, a surfactant, a binder, a lubricant, and the like. With regard to suitable pharmaceutically acceptable carriers and formulations, the composition may preferably be formulated according to each ingredient by using the methods disclosed in Remington's reference. The pharmaceutical composition of the present invention is not particularly limited in formulation, but may be formulated into an injection, an inhalant, an external preparation for skin, an oral ingestant, or the like.

The pharmaceutical composition of the present invention may be orally administered or may be parenterally administered (for example, applied intravenously, subcutaneously, and through the skin, the nasal cavity, or the respiratory tract) according to a desired method, and the administration dose may vary depending on the patient's condition and body weight, severity of disease, drug form, and administration route and period, but may be appropriately selected by those skilled in the art.

The composition of the present invention is administered in a pharmaceutically effective amount. In the present invention, the “pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and other factors well known in the medical field. The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by those skilled in the art.

Specifically, the effective amount of the composition according to the present invention may vary depending on the patient's age, sex, and body weight, and generally, 0.001 to 150 mg of the composition and preferably, 0.01 to 100 mg of the composition, per 1 kg of body weight, may be administered daily or every other day or may be administered once to three times a day. However, since the dosage may be increased or decreased depending on the administration route, the severity of degradation of an ischemic cerebrovascular disease or sequelae thereof, preferably, ischemic stroke or sequelae thereof, the sex, the body weight, the age, and the like, the dosage is not intended to limit the scope of the present invention in any way.

Meanwhile, as another aspect of the present invention, the present invention provides a method for regulating or treating an ischemic cerebrovascular disease or sequelae thereof, the method including: administering the pharmaceutical composition to an individual, and preferably provides a method for regulating or treating ischemic stroke or sequelae thereof, but is not limited thereto.

As used herein, the term “treatment” refers to all actions in which symptoms of an ischemic cerebrovascular disease or sequelae thereof, preferably, ischemic stroke or sequelae thereof are alleviated or beneficially changed by administering the pharmaceutical composition according to the present invention.

As used herein, the “individual” refers to a subject in need of control or treatment of a disease, and more specifically, refers to a mammal such as a human or a non-human primate, a mouse, a rat, a dog, a cat, a horse, and a cow.

Hereinafter, preferred examples for helping the understanding of the present invention will be suggested. However, the following examples are provided only to more easily understand the present invention, and the contents of the present invention are not limited by the following examples.

### EXAMPLES

### Example 1. Experimental Materials and Experimental Methods

### 1-1. Experimental Animals

8-week-old Sprague-Dawley male rats weighing 260 to 280 g were used as experimental animals, and the experimental animals were obtained from the Experimental Animal Center, at Soonchunhyang University (Cheonan). All experimental animals were arbitrarily provided with commercially available feed and water under specific temperature, humidity and lighting conditions (light-dark cycle 12:12, 22±2° C., 55±5%). All animal protocols were approved by the Administrative Panel on Laboratory Animal Care of Soonchunhyang University (permit No. SCH16-0024). In performing experiments related to the present invention, all possible efforts were made to prevent the suffering of the experimental animals and to minimize the number of experimental animals used during the experiments.

### 1-2. Method for Generating Middle Cerebral Artery Ligation (Occlusion) (Induction of Middle Cerebral Artery Occlusion (MCAo))

Rats were anesthetized with 2% isoflurane (2% isoflurane in mixed gas of oxygen and nitrous oxide, 0.3:0.7) and their body temperature was maintained at 37° C. using a heating pad during surgery.

All micro-forceps were disinfected. A tip of 4-0 monofilament (nylon suture, Medtronic plc.) was coated with dental silicone to a thickness of 0.37±0.02 mm, and filament fragments were coated with 0.01% (w/v) poly-L-lysine (Sigma, USA).

The monofilament was inserted into the left common carotid artery (CCA) and advanced toward the internal carotid artery (ICA) to a point 18 mm away from the inserted point to block the origin of the middle cerebral artery (MCA).

Next, a laser Doppler probe (Millwey, Axminste, UK) was fixed to the parietal bone to measure the local cortical blood flow supplied by the middle cerebral artery during surgery and when the flood flow was reduced by 20% from a baseline local cortical blood flow, the occlusion was considered successful.

Next, transient local ischemia was induced by the left middle cerebral artery occlusion for 30 minutes. 30 minutes after the onset of ischemia, the rats were recovered from anesthesia, and the middle cerebral artery was reperfused from the transient ischemia.

To verify the induction of middle cerebral artery ligation (occlusion), it was confirmed whether all rats were able to do the following actions after surgery:

\textcircled{1} The front paw on the other side cannot be extended
\textcircled{2} After neurological deficit, a circling action in the opposite direction on the side where infarction occurs (circling toward contralateral to infarct) cannot be made

The pharmaceutical composition according to the present invention was applied to the induction model of middle cerebral artery ligation (occlusion), and used to perform various behavioral tests, electrophysiology, and immunohisto-chemistry experiments in a specified time course.

An induction model of middle cerebral artery ligation (occlusion) which had no experience with the behavioral tests and the like and normal rats of the same age were used as a control.

### 1-3. Experimental Drug Treatment

The present inventors evaluated the effects of a phosphodiesterase type 5 activity inhibitor and/or a Rho-associated kinase (ROCK) inhibitor on direct damage to the brain or various phenotypic sequelae caused by minor ischemic stroke by applying each of i) sildenafil citrate, ii) GSK429286A and iii) sildenafil citrate and GSK429286A to experimental animals.

More specifically, the following experimental drugs were each intraperitoneally administered 30 minutes before reperfusion after middle cerebral artery occlusion and 30 minutes after reperfusion after middle cerebral artery occlusion.

i) sildenafil citrate (20 mg/kg/saline, Hanmi Parm. Co., Republic of Korea)
ii) GSK429286A (10 mg/kg/DMSO, Sigma, USA)
iii) sildenafil citrate (20 mg/kg/saline) and GSK429286A (10 mg/kg/DMSO)

### 1-4. Measurement of Infarction Volume

To measure the size of the cerebral infarction after the onset of mild ischemic stroke, the heads of the experimental animals were cut, and the brains were removed and cooled at 4° C. to harden the tissue.

Further, 2-mm brain sections were made from 6 or 7 coronal sections from the olfactory bulb to cerebellum.

Next, each of the coronal section brain slices was immersed in a 2% TTC solution at room temperature for 20 minutes.

Finally, images were produced from TTC-stained coronal section brain slices using a model DP72 digital camera and DP2-BSW microscope digital camera software (Olympus, Japan), the area of ischemic brain damage in each brain slice was measured using image analysis software (ImageJ, NIH, USA). TTC staining separates the core area of the cerebral infarct area, a pink damaged brain tissue is classified into the ischemic penumbra which is located around the infarct core area, and a red damaged brain tissue is classified into an undamaged normal brain tissue.

The volume of total cerebral infarction was the sum of the core and the ischemic penumbra, the damaged volume was calculated as an average percentage of the hemispherical volume, and the volume of edema was adjusted with reference to the results of previous studies. Experimental results were provided as mean SEM for different animal groups, and a p-value of <0.01 or <0.05 was considered statistically significant by the Bonferroni's test.

### 1-5. Open Field Test

An open field test is an experiment based on the exploratory behavior of rodents that become more active due to curiosity in a new environment, and the total distance traveled is used as a parameter for evaluating general motor activity.

An open field black box (60×60×40 cm; length, width, height) was placed in an isolated room without clues, and the experimental animals were placed in the central area of the open field box under diffused light. The distance traveled by the experimental animals was recorded and analyzed for 30 minutes using Ethovision® software, which is a PC-based video motion analysis system.

### 1-6. Light-Dark Box Test

A light-dark box (45×27×27 cm; length, width, height) was divided into two compartments, and more specifically, three-fifths was a bright compartment and two-fifths was a dark compartment. Each compartment was connected by a 9×9 cm entrance located on the floor.

By adjusting the lighting in the center of the compartment, the bright compartment and the dark compartment were set to 300 lux and 0-1 lux, respectively, and the experimental animals were placed in the bright compartment and recorded with a video camera for 5 minutes as the experimental animals explored the environment.

### 1-7 Barnes Maze Test

A Barnes maze consisted of a circular platform (122 cm in diameter) with 20 holes around the same distance and elevated 105 cm from the floor. Four different visual cues were placed on each quadrant wall surrounding the platform at a height that makes it easy to observe experimental animals, and one hole (escape hole) in the quadrant area includes an escape chamber under the platform. Further, bedding was added to the escape chamber for the safety of experimental animals. A metronome and bright lighting were used to amplify the anxiety for motivation to find an escape chamber. More specifically, a metronome was used to generate 80 Hz noise and to maintain the illuminance measured at the center of the platform at 300 lux.

During the acclimatization step, the experimental animal was placed in a black cylinder at the center of the platform, and the acclimatization step lasted for 4 days. 10 seconds later, when the cylinder was removed, the experimental animal explored the platform from the center and this activity was recorded by a video camera. In addition, the experimental animals were given 180 seconds to find the escape hole. When the experimental animal entered the escape chamber through the escape hole within 180 seconds, a cover was placed on the hole for 120 seconds to block the light and stop the 80 Hz noise.

Furthermore, when the experimental animal could not find an escape hole, the experimental animal was carefully pulled and guided to the escape hole with an escape chamber to induce learning about the space. The learning was an adaptation step to improve spatial memory to find a way out, and was performed on each experimental animal three times at 15-minute intervals.

During a space acquisition step (probe step) on day 5, the experimental animals were given 90 seconds to find a way out, but the escape chamber was removed. This step was performed only once.

The distance and duration traveled from the quadrant area where the escape chamber connected to the escape hole is located was analyzed by Ethovision® software, and the waiting time to reach the escape chamber connected to the escape hole was manually analyzed.

1-8. Measurement of fEPSP Slope

Field potentials (fEPSP) were recorded at a slightly modified part from CA1 of the hippocampus. After a behavioral test, the animals were anesthetized intraperitoneally using urethane (1.5 g/kg) and placed in a stereotaxic frame. The rectal temperature during surgery was maintained at 37±0.3° C. using a temperature controller (Harvard Instruments, USA).

Next, the scalp was opened and separated, and a hole was made through the skull to introduce electrodes.

The coordinates (in mm) based on bregma (a junction of the coronal suture and the sagittal suture in the skull) are as follows.

i) Recording electrode (to the Schaffer collateral): 4.0 posterior from bregma, 3.0 from centerline to side, 2.5 depth
ii) Stimulating electrode (to the stratum radiatum of CA1): 3.5 posterior from bregma, 2.0 from centerline to side, 3.5 depth from hippocampus.

The depth of the electrodes was finally determined by observing whether the induced response was optimized. The fEPSP was adjusted to about 60% of the maximum response size for testing, and stimuli were generated by a BNC-2110 apparatus (National Instruments, USA) and a Digital Stimulus Isolation unit (Getting Instruments, CA, USA). The response of pyramidal neurons to the Schaffer collateral stimulus was recorded using a P55 A.C. pre-amplifier (3-1000 Hz bandpass, Astro-Med Inc.) and analyzed using WinLTP ver. 2.01 software (WinLTP Ltd.). Further, responses were triggered by a single pulse stimulus and were transmitted at 20 second intervals. A stable baseline was recorded for 30 minutes to 60 minutes.

Long-term potential (LTP) was induced by a strong theta patterned stimulus (sTPS, four trains of 10 bursts of 5 pulses at 400 Hz with a 200-ms inter-burst interval, 15-s inter-train interval). This is because bursts of 400 Hz stimuli (sTPS) are NMDA-receptor dependent and strong LTPs in the CA1 and DG areas are induced by in vivo sTPS.

To analyze changes in fEPSP, the slopes of fEPSP were averaged at 60 second intervals and shown as a percentage of the mean fEPSP slope measured for a reference time of 30 minutes, which was expressed as 100%. The response of fEPSP for the age-matched control was also measured using the same method.

### Example 2. Confirmation of Degree of Damage to Brain Tissue after Stroke During Co-Administration of Sildenafil and ROCK Inhibitor

An experiment was performed to confirm the degree of direct damage to brain tissue due to the onset of stroke when i) sildenafil or the ROCK inhibitor was administered alone, and (6) sildenafil and the ROCK inhibitor were co-administered using MCAo30, which is an acute stroke (cerebral infarction) model. The experiment was performed based on the method described in 1-4.

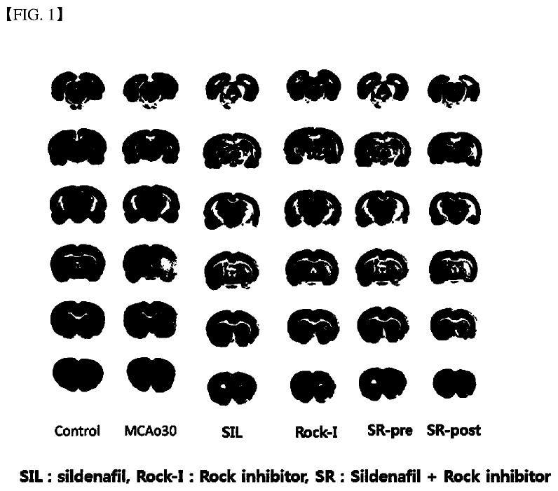

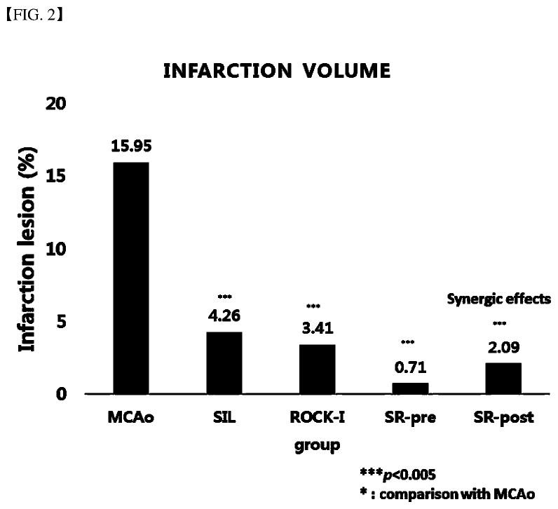

As a result, as illustrated in FIGS. 1 and 2, it was confirmed that when compared to MCAo30 (100% stroke range basis), which is an acute stroke (cerebral infarction) model, the sildenafil-alone administration group had a reduction in damage to brain tissue after the onset of stroke to 26.71%, and the ROCK inhibitor-alone administration group had a reduction in damage to brain tissue after the onset of stroke to 21.28%.

In addition, it was confirmed that the co-administration group of sildenafil and the ROCK inhibitor had a remarkably reduced damage size of brain tissue due to stroke compared to the each alone-administration group in both administration groups before/after reperfusion after middle cerebral artery occlusion (4.5%).

When the experimental results are numerically compared, it can be confirmed that the administration group of the combination therapy of sildenafil and the ROCK inhibitor before reperfusion after middle cerebral artery occlusion exhibits an “effect of a reduction in damage to brain tissue” which is about 6-fold better than the sildenafil-alone administration group, and exhibits an “effect of a reduction in damage to brain tissue” which is about 4.8-fold better than the ROCK inhibitor-alone administration group.

Furthermore, when the experimental results were combined, it could be seen that when the combination therapy of sildenafil and the ROCK inhibitor after the onset of stroke was performed, the effect of a reduction in direct damage to brain tissue due to stroke was maximized, and a synergistic effect occurred in the reduction in damage to brain tissue compared to each single treatment of sildenafil or the ROCK inhibitor.

### Example 3. Confirmation of Whether Loss of Locomotor Activity is Ameliorated when Sildenafil and ROCK Inhibitor are Co-Administered Through Open Field Test

To confirm the degree of loss of locomotor activity, which is one of the sequelae due to stroke when i) sildenafil or the ROCK inhibitor was administered alone and ii) sildenafil and the ROCK inhibitor were co-administered, by using MCAo30 which is an acute stroke (cerebral infarction) model, an open field test was performed, and the locomotor activity was based on the locomotor activity of the normal control (no loss of locomotor activity, 100%). Here, sildenafil and the ROCK inhibitor were co-administered after reperfusion after middle cerebral artery occlusion.

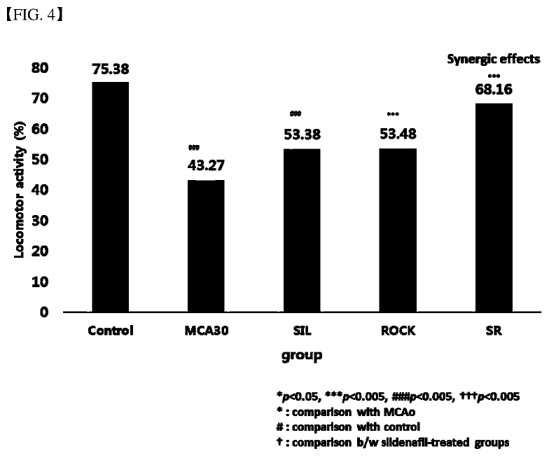

As a result, as illustrated in FIG. 4, it was confirmed that the stroke (cerebral infarction) animal model (MCAo30) had a locomotor activity loss to 57.4% on average, and the results confirmed that the animal model could be used as an animal model of sequelae following stroke (locomotor activity loss sequelae).

Further, it was confirmed that in the case of the sildenafil-alone administration group, the animal model had an improvement in locomotor activity to 70.8%, and in the case of the ROCK inhibitor-alone administration group, the animal model had an improvement in locomotor activity to 71%.

In addition, it was confirmed that compared to the locomotor activity of each alone-administration group of sildenafil or the ROCK inhibitor, the co-administration group of sildenafil and the ROCK inhibitor had a further improvement in locomotor activity of up to 90.4%.

When the experimental results are numerically compared, it can be confirmed that the co-administration group of sildenafil and the ROCK inhibitor exhibits an “effect of an improvement in locomotor activity” which is about 2.5-fold better than the sildenafil-alone administration group, and exhibits an “effect of an improvement in locomotor activity” which is about 2.4-fold better than the ROCK inhibitor-alone administration group.

Furthermore, when the experimental results were combined, it could be seen that when the combination therapy of sildenafil and the ROCK inhibitor is administered during locomotor activity loss following stroke, the effect of ameliorating locomotor activity loss due to stroke was maximized, and a synergistic effect occurred in the amelioration of loss of locomotor activity compared to each single treatment of sildenafil or the ROCK inhibitor.

### Example 4. Confirmation of Whether Anxiety Disorder is Ameliorated when Sildenafil and ROCK Inhibitor are Co-Administered Through Light-Dark Box Test

To confirm the degree of an anxiety disorder, which is one of the sequelae due to stroke when i) sildenafil or the ROCK inhibitor was administered alone and ii) sildenafil and the ROCK inhibitor were co-administered, by using MCAo30 which is an acute stroke (cerebral infarction) model, a light-dark box test was performed, and the degree of anxiety was based on the normal control (no anxiety, 100%). Here, sildenafil and the ROCK inhibitor were co-administered after reperfusion after middle cerebral artery occlusion.

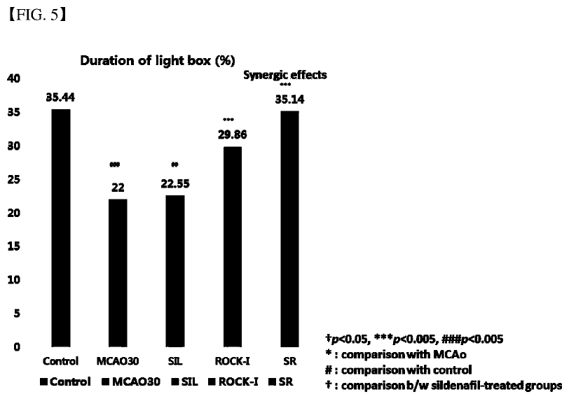

As a result, as illustrated in FIG. 5, it was confirmed that the stroke (cerebral infarction) animal model (MCAo30) had a worsened anxiety level because the time in which rats stayed in a bright place was reduced to 62.08% compared to the normal control.

Further, it was confirmed that the sildenafil-alone administration group had no significant amelioration effect on the worsened anxiety level (anxiety disorder) due to stroke because the time in which rats stayed in a bright place was observed to be 63.63% compared to the normal control, and it was confirmed that the ROCK inhibitor-alone administration group had an amelioration effect on the worsened anxiety level (anxiety disorder) due to stroke because the time in which rats stayed in a bright place was observed to be 84.26% compared to the normal control.

In addition, it was confirmed that the co-administration group of sildenafil and the ROCK inhibitor had a significant amelioration effect on the worsened anxiety level (anxiety disorder) due to stroke because the time spent in a bright place was observed to be 99.2% compared to the normal control, which is almost the same as that of the normal control.

When the experimental results are numerically compared, it can be confirmed that the co-administration group of sildenafil and the ROCK inhibitor exhibits an “effect of ameliorating an anxiety level (anxiety disorder)” which is about 1.7-fold better than the ROCK inhibitor-alone administration group.

### Example 5. Confirmation of Whether Cognitive Function Decline (Long-Term Memory) is Ameliorated when Sildenafil and ROCK Inhibitor are Co-Administered Through Barnes Maze Test

To confirm the degree of cognitive function decline, which is one of the sequelae due to stroke when i) sildenafil or the ROCK inhibitor was administered alone and ii) sildenafil and the ROCK inhibitor were co-administered, by using MCAo30 which is an acute stroke (cerebral infarction) model, a Barnes maze test was performed, and the cognitive function was based on an escape time of the normal control.

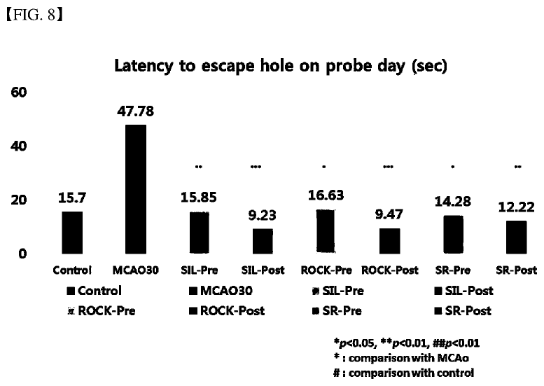

As a result, as illustrated in FIG. 8, it was confirmed that the escape time for finding an escape hole in the stroke (cerebral infarction) animal model (MCAo30) was about 3-fold longer than in the normal control group. The results confirm that memory degradation is one of the most serious sequelae following stroke.

Furthermore, it was confirmed that when sildenafil or the ROCK inhibitor was administered alone, cognitive function decline was significantly ameliorated because the escape times of the sildenafil-alone administration and the ROCK inhibitor-alone administration group were all measured at a level similar to that of the normal control.

Further, it was confirmed that cognitive function decline was significantly ameliorated because the escape time of the co-administration group of sildenafil and the ROCK inhibitor was measured at a level similar to that of the normal control, and in particular, it was confirmed that some individuals exhibited an enhanced cognitive function (enhancement in memory) compared to the cognitive function of the normal control because the escape time was remarkably shortened compared to the normal control.

In addition, it was confirmed that the effect of an improvement in cognitive function was excellent in a drug administration group after reperfusion after middle cerebral artery occlusion because the escape time of a group to which a drug was administered after reperfusion was remarkably significantly shortened compared to groups to which a drug was administered before reperfusion after middle cerebral artery occlusion.

Based on the experimental results, it can be seen that when sildenafil and the ROCK inhibitor are co-administered after reperfusion after middle cerebral artery occlusion, the cognitive function decline due to stroke could be more effectively ameliorated and the cognitive function could be enhanced compared to the normal control.

### Example 6. Confirmation of Whether Synaptic Plasticity is Improved when Sildenafil and ROCK Inhibitor are Co-Administered

An experiment was performed to confirm whether synaptic plasticity was improved when i) sildenafil was administered alone before reperfusion after middle cerebral artery occlusion and ii) sildenafil was administered alone after reperfusion after middle cerebral artery occlusion, by using MCAo30 which is an acute stroke (cerebral infarction) model, the cognitive function was compared to the normal control.

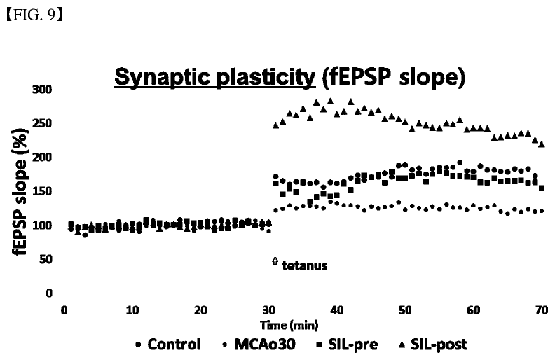

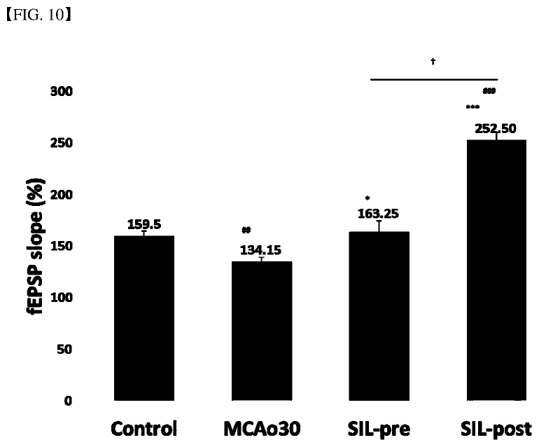

As a result, as illustrated in FIGS. 9 and 10, it was confirmed that in MCAo30 that is a stroke model, the fEPSP slope deteriorated to 84.11%.

However, it was confirmed that in the drug administration group before reperfusion after middle cerebral artery occlusion in the sildenafil-alone administration group, the fEPSP slope was improved to 102.35%, which is at the almost same level as that of the normal control, and in the drug administration group after reperfusion after middle cerebral artery occlusion, the fEPSP slope was improved to 158.31%.

When the experimental results were numerically compared, it was confirmed that in the drug administration group after reperfusion after middle cerebral artery occlusion, the effect of improvement in synaptic plasticity was significantly improved because the drug administration group after reperfusion after middle cerebral artery occlusion had a about 4.06-fold improved fEPSP slope compared to the drug administration group before reperfusion after middle cerebral artery occlusion, and the results coincide with the results in Example 5.

Furthermore, when the experimental results are combined, it can be seen that when single administration treatment of sildenafil is performed after reperfusion after middle cerebral artery occlusion in the stroke (cerebral infarction) animal model, the effect of ameliorating a decrease in synaptic plasticity due to stroke is maximized.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

According to the present invention, it was confirmed that by treatment with sildenafil and a ROCK inhibitor GSK429286A in combination, the effects of a decrease in damage to brain tissue, amelioration of loss of locomotor activity, amelioration of an anxiety disorder or amelioration of cognitive function decline were better than by single treatment with the drug. Thus, since it is considered that by co-administering the sildenafil and GSK429286A, it is possible to enhance the therapeutic effect on stroke and simultaneously ameliorate various sequelae reported to occur in patients due to stroke, it is expected that the present invention can be usefully used for the treatment of various ischemic cerebrovascular diseases including stroke.

1. A method for treating an ischemic cerebrovascular disease or sequelae following the ischemic cerebrovascular disease, the method including administering a pharmaceutical composition to an individual, wherein the pharmaceutical composition comprising a phosphodiesterase type 5 activity inhibitor; and a Rho-associated kinase (ROCK) inhibitor.

2. The pharmaceutical composition of claim 1, wherein the ischemic cerebrovascular disease is selected from the group consisting of ischemic stroke (cerebral infarction), thrombosis, embolism, transient ischemic attack, leukoplakia and small infarction.

3. The pharmaceutical composition of claim 1, wherein the sequelae following the ischemic cerebrovascular disease is any one selected from the group consisting of loss of locomotor activity, an anxiety disorder and cognitive function decline.

4. The pharmaceutical composition of claim 1, wherein the phosphodiesterase type 5 activity inhibitor is any one selected from the group consisting of mirodenafil, sildenafil, vardenafil, tadalafil, udenafil, dasantafil, avanafil and pharmaceutically acceptable salts, solvates and hydrates thereof.

5. The pharmaceutical composition of claim 1, wherein the ROCK inhibitor is any one selected from the group consisting of fasudil, ripasudil, RKI-1447, Y-27632, GSK429286A, Y-30141 and pharmaceutically acceptable salts, solvates and hydrates thereof.

6. The pharmaceutical composition of claim 1, wherein the pharmaceutical composition is for administration before reperfusion after cerebrovascular occlusion, simultaneously with the reperfusion or after the reperfusion.

7. A method for improving a prognosis of an ischemic cerebrovascular disease, the method including administering a pharmaceutical composition to an individual, wherein the pharmaceutical composition comprising a phosphodiesterase type 5 activity inhibitor; and
a Rho-associated kinase (ROCK) inhibitor.

8. A method for enhancing a cognitive function, the method including administering a pharmaceutical composition to an individual, wherein the pharmaceutical composition comprising a phosphodiesterase type 5 activity inhibitor; and
a Rho-associated kinase (ROCK) inhibitor.
